SEQUENCE LISTING (10 sequences)

SEQ ID NO: 1 - protein, 504 aa, Artificial Sequence
  recombinant or laboratory made
MVRLPLQCVLWGCLLTAVHPEHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGGSGGGPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 506 aa, Artificial Sequence
  Recombinant or laboratory made
MVRLPLQCVLWGCLLTAVHPEHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSGGGGPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 3 - protein, 508 aa, Artificial Sequence
  Recombinant or Laboratory made
MVRLPLQCVLWGCLLTAVHPEHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGSGGGGSGGGGSGGPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 4 - protein, 510 aa, Artificial Sequence
  Recombinant or Laboratory made
MVRLPLQCVLWGCLLTAVHPEHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGSGGGGSGGGGSGGGGSGPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 5 - protein, 512 aa, Artificial Sequence
  Recombinant or Laboratory made
MVRLPLQCVLWGCLLTAVHPEHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGSGGGGSGGGGSGGGGSGGPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 6 - protein, 515 aa, Artificial Sequence
  Recombinant or laboratory made
MVSLPRLCALWGCLLTAVHLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGGSGGGGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 7 - protein, 517 aa, Artificial Sequence
  Recombinant or laboratory made
MVSLPRLCALWGCLLTAVHLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGGSGGGGSGGGGGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 8 - protein, 519 aa, Artificial Sequence
  Recombinant or laboratory made
MVSLPRLCALWGCLLTAVHLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGGSGGGGSGGGGSGGGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 9 - protein, 521 aa, Artificial Sequence
  Recombinant or laboratory made
MVSLPRLCALWGCLLTAVHLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGSGGGGSGGGGSGGGGSGGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 10 - protein, 523 aa, Artificial Sequence
  Recombinant or laboratory made
MVSLPRLCALWGCLLTAVHLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKLGGSGGGGSGGGGSGGGGSGGGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

(19) United States
(12) Patent Application Publication
(10) Pub. No.: US 2020/0024326 A1
(43) Pub. Date: Jan. 23, 2020

(54) TNF-TYPE RECEPTOR-LIGAND FUSION PROTEINS AND METHODS

(71) Applicant: NANTBIO, INC., Culver City, CA (US)

(72) Inventors: Kayvan Niazi, Culver City, CA (US); Gard Nelson, Culver City, CA (US); Wendy Higashide, Culver City, CA (US); Philip Liu, Culver City, CA (US)

(21) Appl. No.: 16/440,790

(22) Filed: Jun. 13, 2019

Related U.S. Application Data
(60) Provisional application No. 62/684,938, filed on Jun. 14, 2018.

(51) Int. Cl. C07K 14/705; C12N 15/85; A61K 35/15

(52) U.S. Cl. CPC C07K 14/70578; C12N 15/85; C07K 2319/00; C07K 14/70575; A61K 35/15

(57) ABSTRACT

Self-activating chimeric signaling proteins, and especially chimeric TNF family member ligand-receptor proteins are contemplated. In preferred methods, the chimeric protein comprises an extracellular portion of CD40L that is coupled via a flexible linker to CD40 such that the fusion protein, when expressed in an APC, is capable of folding back on itself and transmits a CD40-mediated signal as if it had been contacted by CD40L located on another cell. Advantageously, cells expressing such chimeric proteins contemporaneously with presentation of an antigen will enhance an immune reaction against the antigen.

This application claims priority to our copending U.S. provisional application with the Ser. No. 62/684,938, filed Jun. 14, 2018.

### FIELD OF THE INVENTION

The field of the invention is fusion proteins, nucleic acids encoding such fusion proteins, and recombinant cells expressing fusion proteins, especially as it relates to CD40/CD40L fusion proteins, 4-1BB/4-1BBL fusion proteins, and Ox40/Ox40L fusion proteins.

### BACKGROUND

The following description includes information that may be useful in understanding the present invention. It is not an admission that any of the information provided herein is prior art or relevant to the presently claimed invention, or that any publication specifically or implicitly referenced is prior art.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

TNF family member receptors such as CD40, 4-1BB, or Ox40, and their respective ligands are known to play a critical role in regulating cellular and humoral immunity. For example, 4-1BB signaling has been implicated with NK cell activation to increase ADCC and IFN-γ secretion, while OX40 signaling is implicated in T cell activation and differentiation. In other examples, CD40 is expressed in various types of immune cells, and predominantly on antigen presenting cells (APCs) such as dendritic cells, macrophages, and B cells. Among other roles, the CD40L/CD40 system is critical to activate and “license” dendritic cells to prime cytotoxic CD8+ T cell responses. Most typically, the CD40 ligand (CD40L) expressed on CD4+ helper T cells engages with CD40 on APCs and so induces APC activation and maturation. The thusly CD40-licensed APCs induce activation and proliferation of antigen-specific CD8+ cytotoxic T cells. A simplified schematic illustrating CD40-CD40L mediated activation of APCs and CD4⁺ and CD8⁺ T cells is depicted in FIG. 1. Notably, without CD40 signaling, interaction of CD8+ T cells with unlicensed APCs induces T cell anergy or triggers formation of regulatory T cells, which may be one of the mechanisms by which tumors persist in a mammal despite presentation of otherwise antigenic peptides.

In view of the critical role of CD40, numerous attempts have been undertaken to employ CD40 activation in therapy. For example, CD40 signaling can be effectively triggered using agonistic antibodies or soluble CD40L (e.g., Int Rev Immunol 2012, 31:246-266). However, such approach is often limited by systemic toxicity (e.g., J Clin Oncol 2007, 25:876-883; Science 2012, 331:1612-1616). More recently, it was discovered that efficacy of CD40 signaling is dependent on the multimerization or trimerization of CD40. On that basis, a multi-trimeric fusion construct of CD40L and the gp100 tumor antigen was prepared, and was shown to activate dendritic cells and to enhance survival in a B16-F10 melanoma DNA vaccine model (see e.g., Vaccine. 2015 Sep. 11; 33(38): 4798-4806).

In still other known attempts to activate APCs, a chimeric polypeptide was constructed that consisted of the signal transduction domain of CD40 fused to a 50-100 amino acid spacer, which was in turn fused to the binding and trimerization domain of CD40L as disclosed in WO 00/063395. Similarly, a chimeric polypeptide was constructed that consisted of the signaling domain of CD40 fused to a type 2 receptor transmembrane domain that was fused to the binding and trimerization domain of CD40L as disclosed in WO 02/036769. The constructs were then expressed in tumor cells and so transfected cells were implanted in mice. No therapeutic effect, however, was provided in these references. In still other known methods, a chimeric protein was expressed in antigen presenting cells where the chimeric protein consisted of CD40 cytoplasmic region that was fused to a FK506 ligand binding region and a myristoylation membrane targeting region as is disclosed in U.S. Pat. No. 7,404,950. Similarly, a fusion protein with a multimeric ligand binding region and a CD40 portion lacking the extracellular domain was described in U.S. Pat. No. 8,999,949. While such constructs may provide at least some increased activity in vitro, they are prone to be antigenic upon administration to a mammal.

Therefore, while various manners of modulating TNF family member receptor/ligand signaling are known in the art, all or almost all of them suffer from one or more disadvantages. Thus, there is still a need for improved modulation of TNF family member receptor/ligand signaling.

### SUMMARY OF THE INVENTION

The inventive subject matter is directed to various chimeric constructs comprising a TNF family member ligand and a TNF family member receptor, and nucleic acids encoding same, as well as to cell transfected with such nucleic acids and methods of treating cancer and viral infections. In particularly preferred aspects, a CD40L-CD40 fusion protein is constructed and expressed in an APC wherein the fusion protein is capable of folding back on itself to so transmit a CD40-mediated signal as is it was activated by a separate cell with a CD40L (e.g., CD4+ T cell). Similarly, in further contemplated aspects, 4-1BB ligand/4-1BB and Ox40L/Ox40 fusion proteins are contemplated and expressed in suitable immune competent cells.

In one aspect of the inventive subject matter, the inventors contemplate a chimeric protein that includes in sequence from N- to C-terminus, an extracellular portion of CD40L that is coupled to a flexible linker that is coupled to CD40. In especially contemplated aspects, the chimeric protein also comprises a leader peptide that is coupled to the N-terminus of the extracellular portion of CD40L.

Most preferably, but not necessarily, the extracellular portion of CD40L is a human extracellular portion of CD40L and the CD40 is a human CD40, and/or the flexible linker has a length of between 4-25 amino acids (e.g., including a (GₙS)ₓ motif with n and x independently between 1-5). Most typically, the CD40 will lack a signal sequence as compared to a full length sequence. Therefore, and among other contemplated options, the chimeric protein may have a sequence of any one of SEQ ID NO: 1-10.

In a further contemplated aspect of the inventive subject matter, a recombinant expression cassette will include a promoter that is operably coupled to a segment that encodes the chimeric protein as contemplated herein. Where desired, the recombinant expression cassette may also include a second segment that encodes a cytokine and/or at least a portion of at least one of a tumor associated antigen (TAA), a tumor specific antigen (TSA), and a tumor and patient specific neoepitope. As will be readily appreciated, the recombinant expression cassette may be an RNA, or may be part of a viral expression vector (which may or may not be encapsulated in a viral particle).

Therefore, in still further contemplated aspects, the inventors contemplate a recombinant cell that is transfected with a recombinant expression cassette as contemplated herein. Most typically, the cell is an antigen presenting cell (e.g., dendritic cell), and/or the cell is transiently transfected.

Viewed from a different perspective, the inventors also contemplate a method of enhancing an immune reaction against an antigen that includes a step of transfecting an antigen presenting cell with a nucleic acid construct comprising a recombinant expression cassette as presented herein, and a further step of contacting the transfected cell with the antigen or expressing the antigen in the transfected cell. Upon contact or expression, the transfected cell is then contacted with a CD4+ T cell and/or a CD8+ T cell.

For example, contemplated antigens are tumor and patient specific neoepitopes, or at least a portion of a tumor associated antigen (TAA) or a tumor specific antigen (TSA). It is further generally contemplated that the step of transfecting is performed ex vivo, and that the steps of contacting are performed in vivo. Therefore, the immune reaction against the antigen may be an immune reaction against a tumor or against a virus (e.g., HIV) in an individual.

Consequently, the inventors also contemplate a method of treating a tumor in an individual that includes the steps of transfecting an antigen presenting cell of the individual with a recombinant expression cassette as presented herein, and a further step of contacting the transfected cell with a tumor antigen or expressing the tumor antigen in the transfected cell. Upon contact or expression, the transfected cell is then contacted with a CD4+ T cell and/or a CD8+ T cell of the individual.

As noted before, it is typically contemplated that the step of transfecting is performed ex vivo, and wherein the steps of contacting are performed in vivo. Moreover, it is contemplated that the tumor antigen is a tumor and patient specific neoepitope, or at least a portion of a tumor associated antigen or a tumor specific antigen. In preferred aspects, the antigen presenting cell is a dendritic cell, and the recombinant expression cassette is an mRNA or part of an adenovirus.

Therefore, the inventors also contemplate use of a chimeric protein and/or use of a recombinant cell as presented herein to treat a cancer or viral infection.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a simplified schematic illustrating CD40-CD40L mediated activation of APCs and CD4⁺ and CD8⁺ T cells.

FIG. 2 depicts several views of predicted structures of an exemplary fusion protein contemplated herein.

FIG. 3 depicts results for cells expressing exemplary fusion proteins contemplated herein.

FIG. 4 depicts exemplary results demonstrating that the constructs are operable in diverse species (murine).

FIG. 5 depicts exemplary results demonstrating that the constructs result in secretion of IL-8 in selected cell lines.

FIG. 6 depicts exemplary results demonstrating that the constructs are operable across diverse species.

FIG. 7 depicts exemplary results for surface expression in 293T cells of the constructs presented herein.

FIG. 8 depicts exemplary results for surface expression in B16F10 cells of the constructs presented herein.

FIG. 9 depicts exemplary results for comparison of 293T cells transfected with CD40 and subsequent stimulation with soluble CD40L versus contemplated constructs.

FIG. 10 depicts exemplary results for cytokine production by human (293T) and mouse (B16F10) cells transfected with human/mouse constructs.

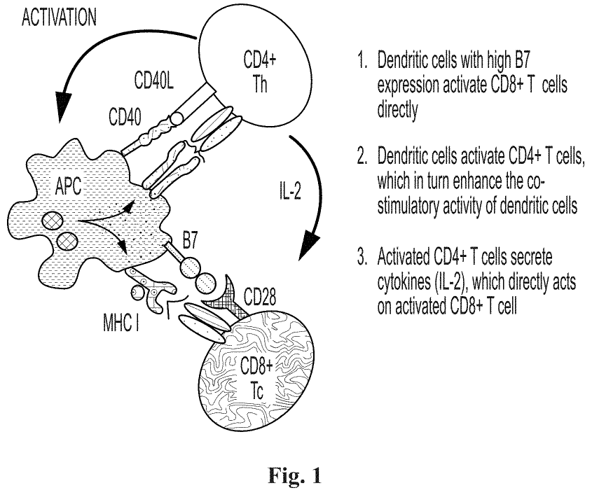

### DETAILED DESCRIPTION

The inventors have now discovered that an immune response to an antigen can be tailored in a desired direction (i.e., enhanced or dampened) by interference with CD40 signaling events at an antigen presenting cell. For example, as described in more detail below, the inventors have constructed and expressed on an APC a self-activating CD40 signaling protein that was capable of folding back on itself and so transmit a CD40-mediated signal into the APC as if it had been contacted by another cell expressing CD40L (e.g., CD4+ T cell). Likewise, the inventors also contemplate modulation of T cell and NK cell activities by expression of the fusion proteins in various immune competent cells (e.g., APCs, NK cells, T cells).

In this context it should be noted that CD40, a type-1 membrane protein in which the N terminus resides on the outside of the cell, is considered a master switch (e.g., on dendritic cells) while CD40L (e.g., located on CD4 T cells) is a type 2 membrane protein where the C terminus is located on the outside of the cell. CD40 like many other members of the TNF family, needs to trimerize to effect signaling at CD40, which is accomplished by interaction with CD40L that has a trimerization domain. Such activation requirement has advantageously been exploited by the inventors by modification of a chimeric CD40 molecule that is coupled to its own CD40L (with trimerization domain) via a linker to so attain proper binding of the CD40 portion to the CD40L portion while allowing trimerization of CD40L portion.

Consequently, where expressed in an antigen presenting cell, the chimeric proteins will necessarily trimerize and so effect CD40 signaling without the need for another cell (typically a CD4+ T cell) to deliver the CD40L. Most preferably, the APC will also express or be exposed to an antigen of choice and therefore present a portion of the antigen on the MHC system. As will be readily appreciated, such APC will be effective in enhancing an immune response, even in the absence (or reduced presence) of CD4+ T cells, which is of significant importance in infections with a pathogen that destroys or reduces CD4+ T cells such as the HIV virus. Viewed from yet another perspective, it should be appreciated that an immune reaction can be enhanced or down-regulated in a tailored antigen specific fashion by co-presentation of the chimeric protein with at least a portion of the antigen on the MHC presentation system. For immune stimulation against a specific antigen, trimerization of the chimeric protein is effected (as shown below). Conversely, for down-regulation of an immune response against a specific antigen, trimerization of the chimeric protein is reduced or inhibited. Likewise, where the TNF family member ligand is 4-1BB ligand or Ox40 ligand, the TNF family member receptor is 4-1BB or Ox40. As these proteins share common structural motifs and activation patterns, it should be recognized that the teachings presented herein with regard to CD40L/CD40 equally apply to 4-1BBL/4-1BB and Ox40L/Ox40.

Therefore, it should be appreciated that such constructs are also particularly relevant to vaccines and other immune stimulating compositions (especially cancer vaccines) where the trimerization concept is transposed onto other TNF family members like 4-1BB, OX40, etc. to activate cells in a desired manner through gene expression. Therefore, contemplated systems and methods are also suitable for use beyond APCs, and especially contemplated uses include those with NK cells and their derivatives (e.g., NK-92, aNK, haNK, tank, etc.), T cells and their derivatives (e.g., CAR-T, TCR-T, TIL-T, etc.), B cells, and so forth. Therefore, while the below discussion provides examples and contemplations for CD40 and CD40L, it should be appreciated that the inventive subject matter also applies to other TNF family members like 4-1BB, OX40, etc.

For example, and with respect to CD40, it is contemplated that all variants of CD40 are deemed suitable for use herein. However, particularly suitable CD40 variants include human and other mammalian forms of CD40. There are numerous such sequences known in the art, and all of these are deemed suitable for use herein (see e.g., uniprot sequence database). In most typical embodiments, but not necessarily, the CD40 signal peptide is removed in contemplated constructs and replaced with an upstream portion that includes a linker and the CD40L portion. Moreover, it is typically preferred that for activating chimeric constructs, the CD40 will retain its intracellular activation domain. On the other hand, where down-regulation is desired, the CD40 will have a truncated intracellular portion lacking a (functional) activation domain.

Most typically, the particular CD40 will be selected to match the species (e.g., human CD40 for human APC) in which it is being used. Moreover, it should also be appreciated that numerous modifications may be implemented to achieve a desired purpose. For example, the intracellular activation domain may be present in multiple copies, or be partially or entirely deleted. In other examples, one or more amino acids may be added as a tag for identification via IHC. In still further examples, one or more amino acids may be exchanged (especially at the N-terminus) to increase the protein half life time. In less preferred aspects, it is also contemplated that the transmembrane domain of CD40 may be replaced with another transmembrane domain.

Likewise, contemplated CD40L sequences may vary considerably, and it is contemplated that all variants of CD40L are deemed suitable for use herein. However and as already noted above, particularly suitable CD40L variants include human and other mammalian forms of CD40L. There are numerous such sequences known in the art, and all of these are deemed suitable for use herein (see e.g., uniprot sequence database). In most typical embodiments, but not necessarily, the CD40L will include its native signal peptide, however, other signal peptides may also be included where desired. Moreover, for activating chimeric constructs, the CD40L will retain its trimerization domain. On the other hand, where down-regulation is desired, the CD40L may have a truncated trimerization domain or other domain that has sufficient steric hindrance to disrupt trimerization.

Most typically, the particular CD40L will be selected to match the species (e.g., human CD40 for human APC) in which it is being used. Moreover, it should also be appreciated that numerous modifications may be implemented to achieve a desired purpose. For example, the trimerization domain may be optimized to increase affinity, or be partially or entirely deleted. In still further examples, one or more amino acids may be exchanged (especially at the N-terminus) to increase the protein half life time.

As will be readily appreciated, suitable linkers will typically be chosen such that the CD40 and CD40 portions will have sufficient mobility relative to each other to all selective binding. Therefore, and especially for activating chimeric molecules, the linker will be a polypeptide that has a length of between 4-30 amino acids with low or no immunogenicity. However, particularly preferred linkers will be GS-type linkers with a length of between 4-25, and most preferably between 15-17 amino acids. There are numerous alternative linkers known in the art, and all of them are deemed suitable for use herein (see e.g., Adv Drug Deliv Rev 2013 Oct. 15; 65(10): 1357-1369).

Constructs are therefore contemplated that include the CD40L portion, the linker, and the CD40 portion in a single polypeptide, and exemplary chimeric constructs are shown in SEQ ID Nos:1-10.

Of course, it should be appreciated that these constructs are in most cases not delivered as a polypeptide to a cell, but that a cell, and most typically an APC, is transfected with a nucleic acid having an expression cassette that encodes the chimeric protein. Therefore, contemplated nucleic acids include those that have a promoter (constitutive or inducible) that controls the expression of the nucleic acid sequence that encodes the chimeric protein. As the protein has a transmembrane portion, the construct will typically have a signal sequence (optionally cleavable) that directs the chimeric protein to the cell surface.

Most typically, the recombinant nucleic acid may be a DNA that may be integrated into the host genome of the APC or that may persist as an extrachromosomal unit. For example, suitable DNA constructs may be linear or circular constructs and may be configured as an expression vector, and particularly as a viral expression vector that can be delivered into the cell via viral infection. Among other options, the viral vector may be an adenoviral vector, and especially an AdV vector with deleted E1 and E2b genes. Alternatively, the nucleic acid may also be RNA, and especially an mRNA or self-replicating RNA to limit the persistence of the recombinant payload.

In further preferred aspects of the inventive subject matter, the host cell (typically the APC) may be transfected with a recombinant nucleic acid that also includes a segment that will encode at least one of a TAA, TSA, and a neoepitope or polytope (each of which may be on the same or different nucleic acid). Advantageously, such transfection will deliver at the same time the chimeric protein along with the specific antigen. On the other hand, the transfected cell may also be exposed to the TAA, TSA, neoepitope, and/or polytope and so take up and process the antigen for presentation of the MHC complexes. In this context, it should be recognized that the recombinant TAA, TSA, neoepitope, and/or polytope may have a trafficking signal that directs the TAA, TSA, neoepitope, and/or polytope to the MHC-I and/or MHC-II complex. However, it is typically preferred that the trafficking is at least to the MHC-II complex.

In still further contemplated aspects, the recombinant nucleic acid also include a segment that encodes one or more cytokines, and especially immune stimulatory cytokines (e.g., IL-2, IL-15, IL-17, IL-21) for increasing an immune response, or a down-regulating cytokine (e.g., IL-10, TGFβ) to dampen an immune response.

Contemplated cells for transfection typically include all types of antigen presenting cells, however, it is particularly preferred that the transfected cell is a dendritic cell or a macrophage. In still further contemplated aspects, the cell for transfection is preferably an autologous APC relative to the patient, or an MHC-matched APC. In less preferred aspects, heterologous APC are also contemplated. Moreover, it should be noted that the cells may be irradiated before administration to reduce persistence, and the person of ordinary skill in the art will be well apprised of the suitable dosages and radiation sources.

Most preferably, cells will be transfected in vitro, cultured as appropriate/needed, and then administered to the patient. Alternatively, it is contemplated that the cells may also be transfected in vivo using a therapeutic virus that includes the recombinant viral expression system.

### EXAMPLES

The inventors have used crystal structures of CD40, CD40L, CD40/CD40L complexes to determine a range of linker lengths that could tether the two protein portions together while at the same time maintaining their functionality. To that end, five linkers of varying length were modeled and recombinantly expressed, and several of the fusion proteins were tested.

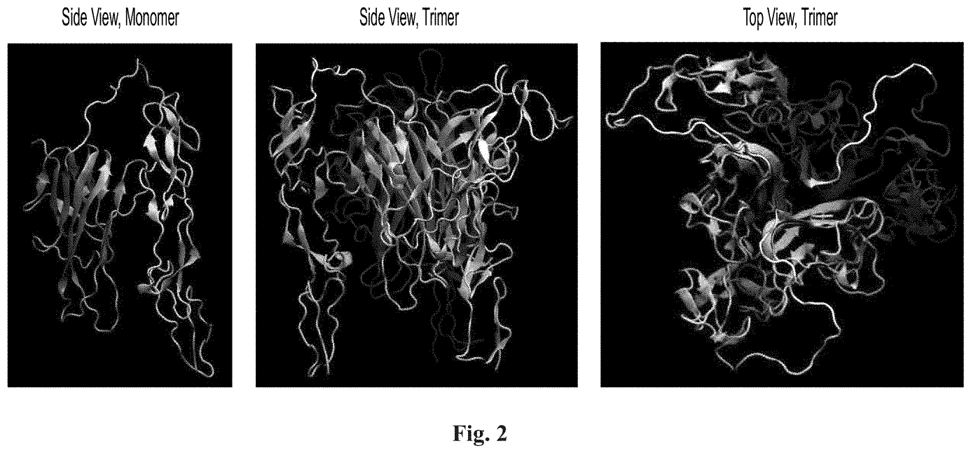

FIG. 2 depicts exemplary models of the 16-mer linker bearing fusion protein in which the left panel shows a predicted side view of the chimeric protein monomer, in which the middle panel depicts a predicted side view of the trimer, and in which the right panel depicts a predicted top view of the trimer. As can be readily seen from the panels, the linker affords sufficient steric mobility to allow binding of CD40L to CD40, and to allow trimerization of the chimeric protein.

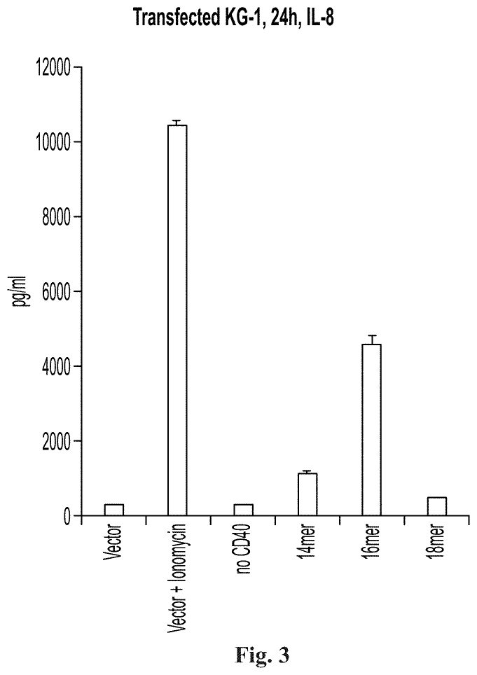

To determine whether these constructs would also exhibit biological effect of immune competent cells, KG-1 cells (myeloid cell line) were transfected with constructs having different linker lengths. These cells transfect at a rate of about 30-50%. Therefore, a response with the linker constructs compared to small molecule activation of the cells with ionomycin is readily observable with respect to cytokine production (here IL-8). FIG. 3 provides exemplary data for human cells transiently transfected with CD40L-Linker-CD40 constructs with varying linker lengths. As can be taken from the data, some of the chimeric constructs triggered substantial activity in the transfected cells, indicating that a linker length of about 16 amino acids was most effective in signaling.

KG-1 cells were transfected via electroporation using BioRad Gene Pulser II, with 3 pulses (200 ohms, 25 μf, 0.26 kV), and cultured in growth medium (Iscove's Modified Dulbecco's Medium supplemented with 20% fetal bovine serum) for 16 hours. The transfected cells are washed to remove residual cytokines that may have resulted from the electroporation process, and cultured in fresh medium in a 96 well tissue culture plate at 20,000 cells per well. The cells are cultured for an additional 24 hours, and the supernatant was harvested. Cytokines levels in the supernantants were determined using Cytometric Bead Array specific for human IL-1β, MCP-1 and IL-8 according to the manufacturer's recommended protocol; however, only IL-8 demonstrated any changes.

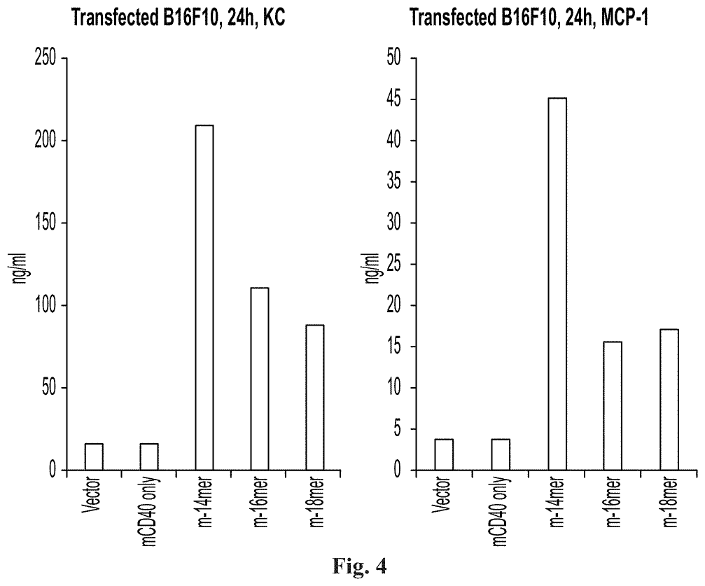

Mouse CD40L/CD40 fusion proteins: To determine whether the concept of self-ligating CD40/CD40L fusion constructs can be expanded to other species, a parallel set of constructs encoding the mouse versions of these proteins was produced and tested in the mouse B16F10 melanoma cell line for activity (FIG. 4). Similar results were obtained in this parallel system indicating the system is likely to be expandable to other CD40 sequences and even other TNF family members. As can be taken from the data, some of the chimeric constructs triggered substantial activity in the transfected cells both (KC and MCP-1), indicating that a linker length of either 14 or 16 amino acids were effective in signaling, whereas, the 18 amino acid linker did not elicit a response.

These transfections were performed as follows. Mouse melanoma cell line B16F10 were transfected with the mouse CD40/CD40L fusion protein constructs using Lipofectamine 2000 according to the manufacturer's recommended protocol. The cells were rested for 18 hours, washed to remove residual cytokine and cultured in fresh growth medium (Dulbecco's Minimum Essential Medium supplemented with 10% fetal bovine serum) in a 96 well tissue culture plate at 50,000 cell per well for an additional 24 hours. Following incubation, the supernatant was harvested and the levels of mouse IL-1β, MCP-1 and KC were determined using cytometric bead array, according to the manufacturer's recommended protocols. The fusion proteins with the 14 and 16 amino acid linker demonstrated a response as measured by production of KC and MCP-1.

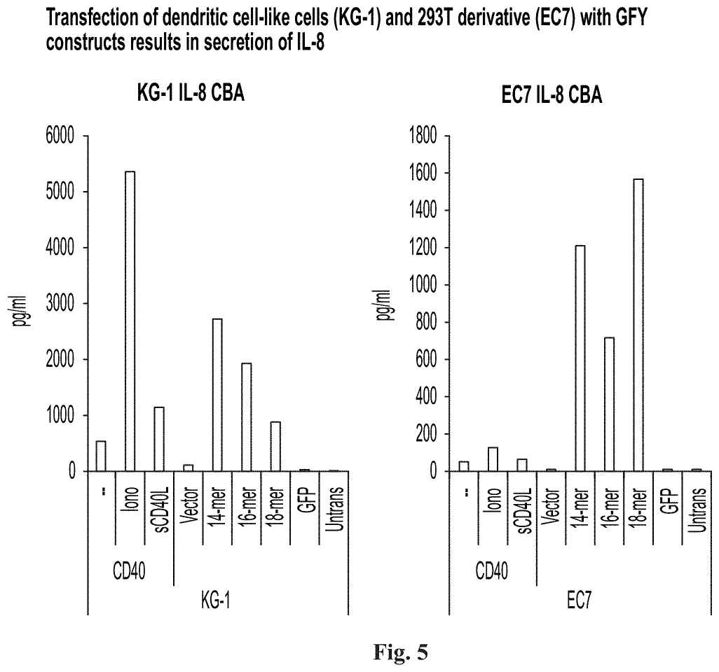

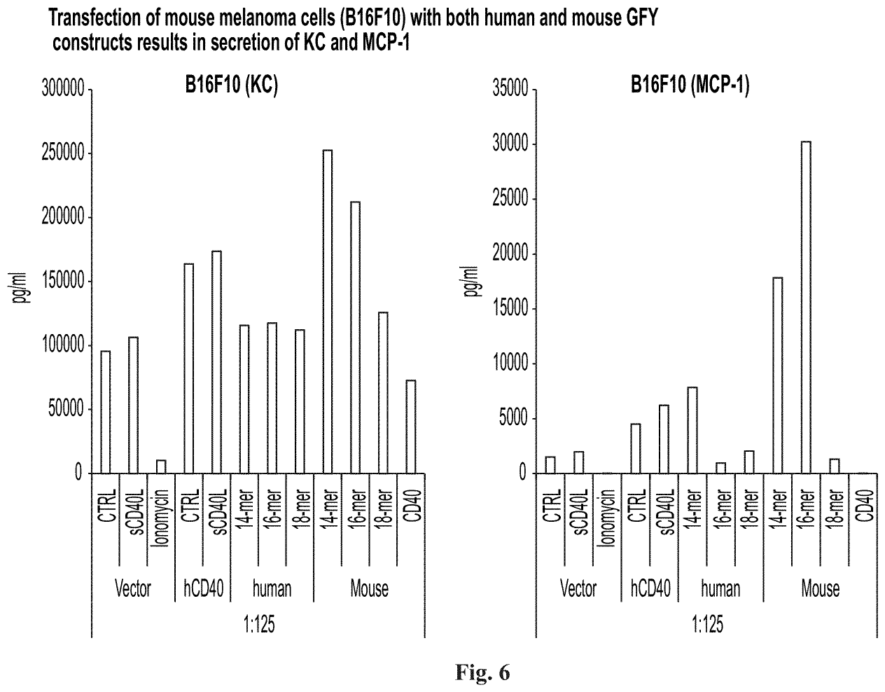

Using substantially same protocols as described above, the inventors further investigated whether transfection of dendritic cell-like cells (KG-1) and 293T derivative (EC7) with the chimeric constructs would result in secretion of IL-8. As can be taken from FIG. 5, both cell lines had significant IL-8 secretion with all variants tested. To further test whether the constructs could operate across species boundaries, the inventors also tested whether transfection of mouse melanoma cells (B16F10) with both human and mouse constructs would result in secretion of the chemokines KC and MCP-1. As can be readily seen from FIG. 6, the chemokines were secreted even where the chimeric construct was not from the same species.

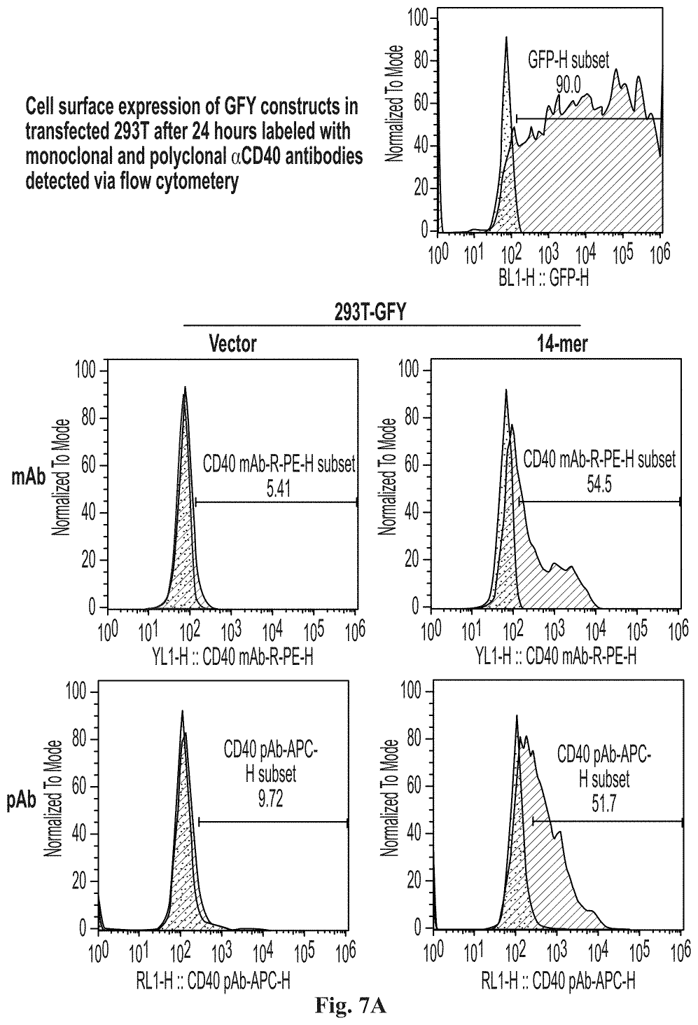

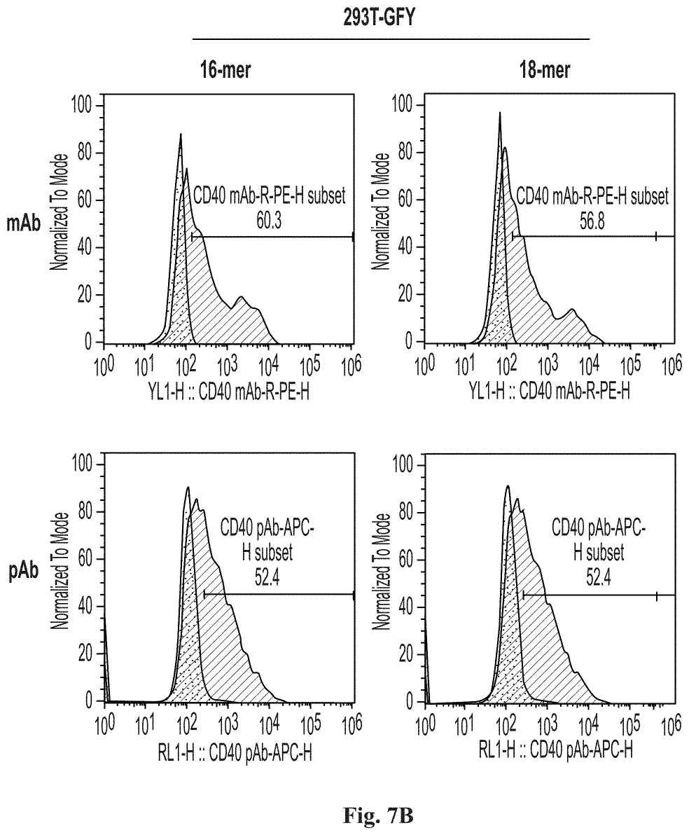

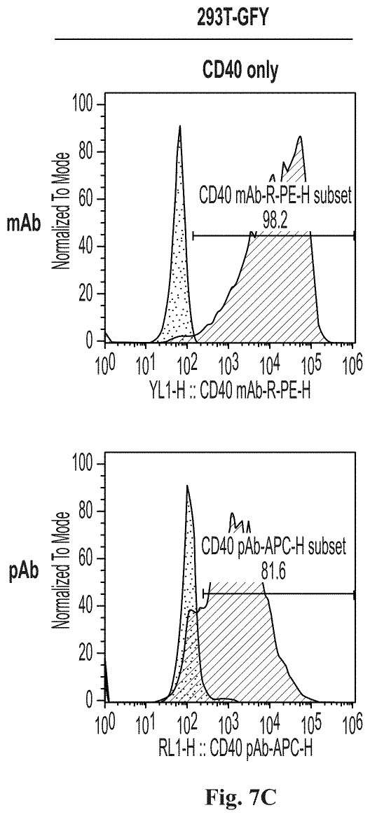

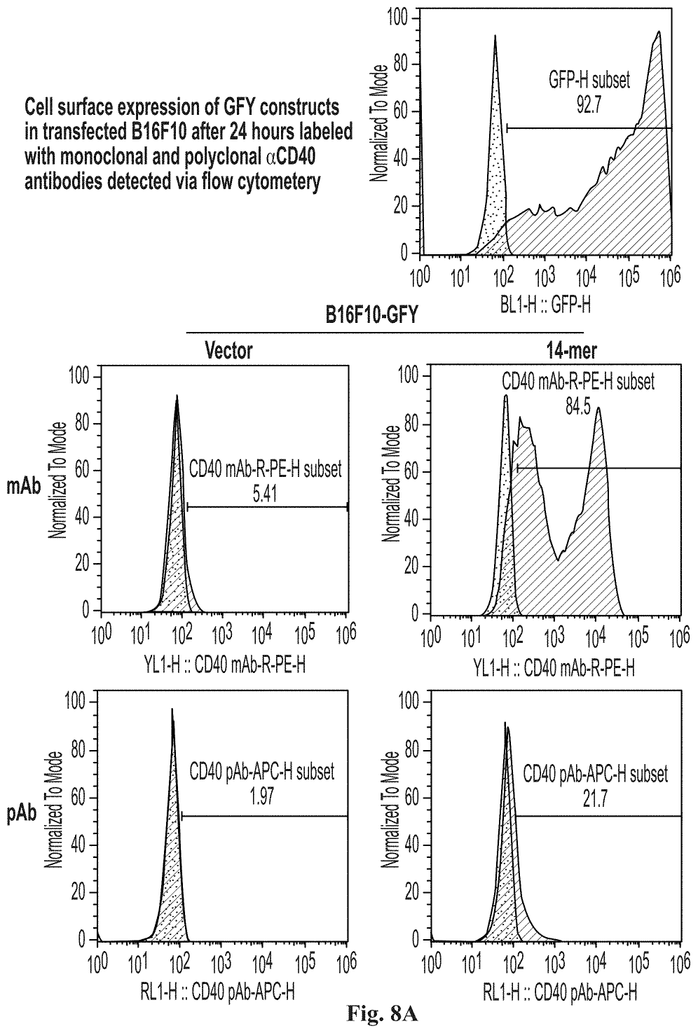

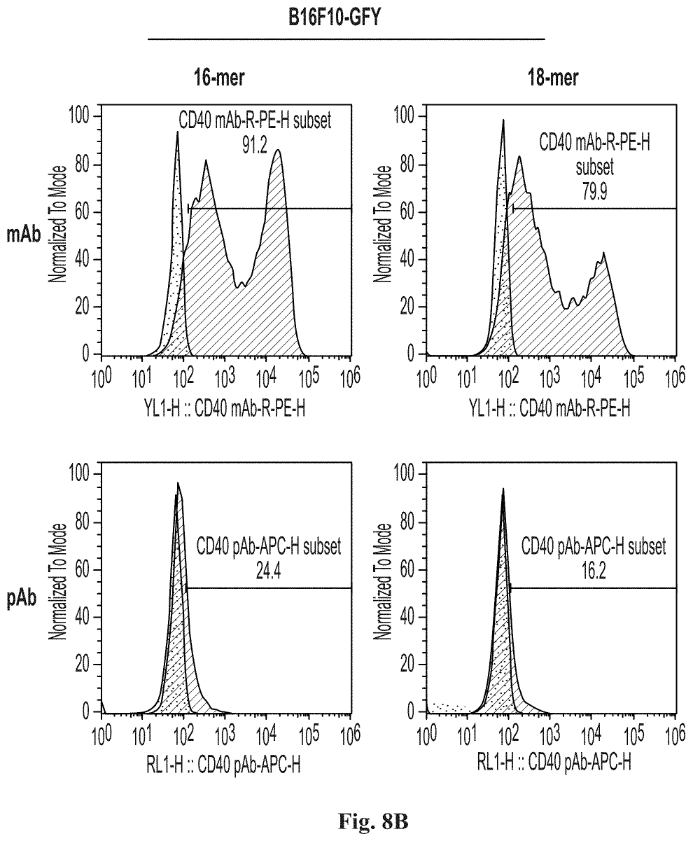

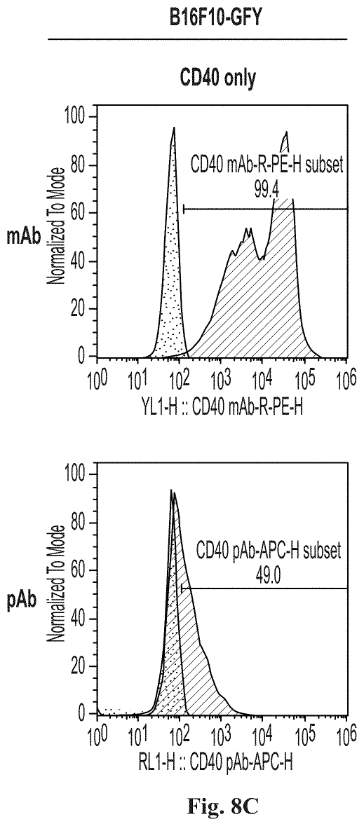

To ascertain expression of the chimeric constructs in human (293T) and murine (B16F10) cells, the cells were transfected and after 24 hours labeled with monoclonal or polyclonal antibodies. The results for these experiments are shown in FIGS. 7 and 8, respectively. As is readily apparent, surface expression was confirmed across both cell lines for all constructs.

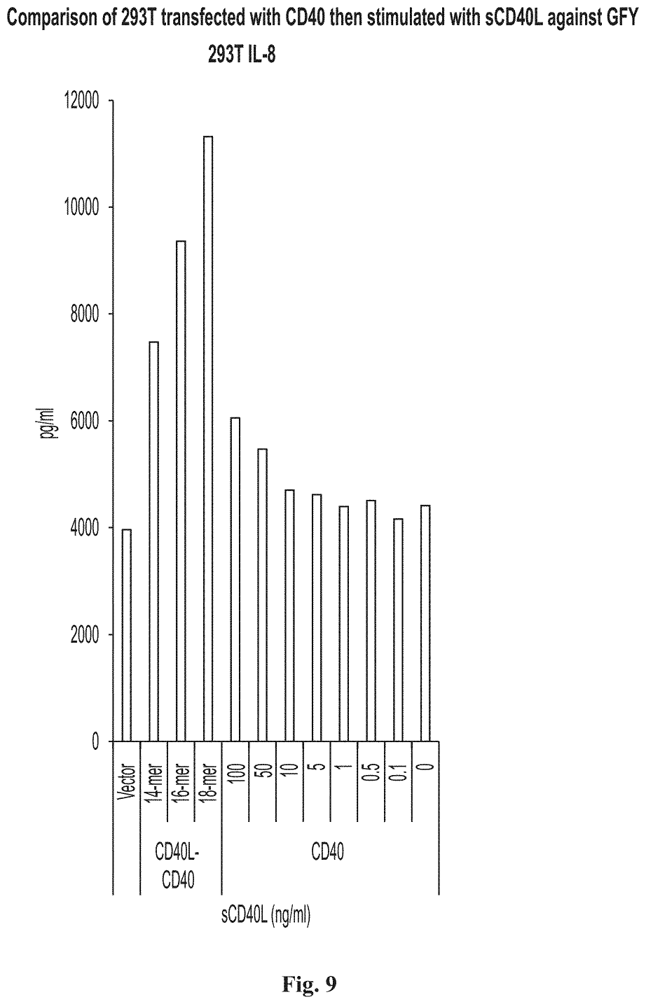

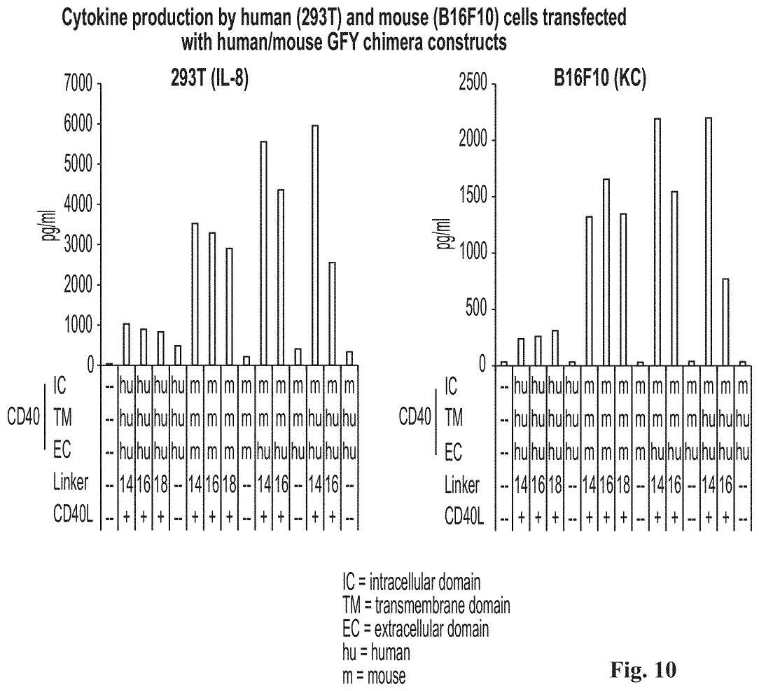

Functionality of the chimeric constructs was tested against 293T transfected with CD40 which were subsequently stimulated with sCD40L, and exemplary results are shown in FIG. 9. Notably, when measuring IL-8 secretion, the chimeric constructs had superior activation as compared to soluble CD40 ligand. Finally, the inventors prepared contemplated constructs in a manner that used mouse and human sequence elements for the CD40 domain of the fusion protein. Therefore, at least some of the fusion proteins were also chimeric with respect to origin of the intracellular (IC), Transmembrane™, or extracellular (EC) domain. Remarkably, as is shown in FIG. 10, chimeric constructs in human cells using human EC had significantly higher activity in human cells, even where murine IC and TM segments were used. Similarly, the human EC was also superior in such constructs in murine cells.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

### EXEMPLARY SEQUENCES

In the following, the CD40L signal sequence is underlined, the CD40L extracellular domain is in normal font, the 12-20 amino acid linker is in bold typeface, and CD40 (minus signal peptide) is in italics.

All constructs were based on the following Uniprot sequences:

human CD40: www.uniprot.org/uniprot/P25942
human CD40L: www.uniprot.org/uniprot/P29965
mouse CD40: www.uniprot.org/uniprot/P27512
mouse CD40L: www.uniprot.org/uniprot/P27548

(CD40/CD40L + 12mer linker)
>SEQ ID.NO: 1
MVRLPLQCVLWGCLLTAVHPEHRRLDKIEDERNLHEDFVFMKTIQRCNTGERSLSLLNC
EEIKSQFEGFVKDIMLNKEETKKENSFEMQKGDQNPQIAAHVISEASSKTTSVLQWAEK
GYYTMSNNLVTLENGKQLTVKRQGLYYIYAQVTFCSNREASSQAPFIASLCLKSPGRFE
RILLRAANTHSSAKPCGQQSIHLGGVFELQPGASVFVNVTDPSQVSHGTGFTSFGLLKLG
GGSGGGGSGGGPPTACREKQYLINSQCCSLCQPGQKLVSDCTEFTETECLPCGESEFLDT
WNRETHCHQHKYCDPNLGLRVQQKGTSETDTICTCEEGWHCTSEACESCVLHRSCSPGFGV
KQIATGVSDTICEPCPVGFFSNVSSAFEKCHPWTSCETKDLVVQQAGTNKTDVVCGPQDRLR
ALVVIPIIFGILFAILLVLVFIKKVAKKPTNKAPHPKQEPQEINFPDDLPGSNTAAPVQETLHGC
QPVTQEDGKESRISVQERQ
(CD40/CD40L + 14mer linker)
>SEQ ID.NO: 2
MVRLPLQCVLWGCLLTAVHPEHRRLDKIEDERNLHEDFVFMKTIQRCNTGERSLSLLNC
EEIKSQFEGFVKDIMLNKEETKKENSFEMQKGDQNPQIAAHVISEASSKTTSVLQWAEK
GYYTMSNNLVTLENGKQLTVKRQGLYYIYAQVTFCSNREASSQAPFIASLCLKSPGRFE
RILLRAANTHSSAKPCGQQSIHLGGVFELQPGASVFVNVTDPSQVSHGTGFTSFGLLKLG
GGGSGGGGSGGGGPPTACREKQYLINSQCCSLCQPGQKLVSDCTEFTETECLPCGESEFL
DTWNRETHCHQHKYCDPNLGLRVQQKGTSETDTICTCEEGWHCTSEACESCVLHRSCSPGF
GVKQIATGVSDTICEPCPVGFFSNVSSAFEKCHPWTSCETKDLVVQQAGTNKTDVVCGPQDR
LRALVVIPIIFGILFAILLVLVFIKKVAKKPTNKAPHPKQEPQEINFPDDLPGSNTAAPVQETLH
GCQPVTQEDGKESRISVQERQ
(CD40/CD40L + 16mer linker)
>SEQ ID.NO: 3
MVRLPLQCVLWGCLLTAVHPEHRRLDKIEDERNLHEDFVFMKTIQRCNTGERSLSLLNC
EEIKSQFEGFVKDIMLNKEETKKENSFEMQKGDQNPQIAAHVISEASSKTTSVLQWAEK
GYYTMSNNLVTLENGKQLTVKRQGLYYIYAQVTFCSNREASSQAPFIASLCLKSPGRFE
RILLRAANTHSSAKPCGQQSIHLGGVFELQPGASVFVNVTDPSQVSHGTGFTSFGLLKLG
GGSGGGGSGGGGSGGPPTACREKQYLINSQCCSLCQPGQKLVSDCTEFTETECLPCGESE
FLDTWNRETHCHQHKYCDPNLGLRVQQKGTSETDTICTCEEGWHCTSEACESCVLHRSCSP
GFGVKQIATGVSDTICEPCPVGFFSNVSSAFEKCHPWTSCETKDLVVQQAGTNKTDVVCGPQ
DRLRALVVIPIIFGILFAILLVLVFIKKVAKKPTNKAPHPKQEPQEINFPDDLPGSNTAAPVQET
LHGCQPVTQEDGKESRISVQERQ
(CD40/CD40L + 18mer linker)
>SEQ ID.NO: 4
MVRLPLQCVLWGCLLTAVHPEHRRLDKIEDERNLHEDFVFMKTIQRCNTGERSLSLLNC
EEIKSQFEGFVKDIMLNKEETKKENSFEMQKGDQNPQIAAHVISEASSKTTSVLQWAEK
GYYTMSNNLVTLENGKQLTVKRQGLYYIYAQVTFCSNREASSQAPFIASLCLKSPGRFE
RILLRAANTHSSAKPCGQQSIHLGGVFELQPGASVFVNVTDPSQVSHGTGFTSFGLLKLG
SGGGGSGGGGSGGGGSGPPTACREKQYLINSQCCSLCQPGQKLVSDCTEFTETECLPCGE
SEFLDTWNRETHCHQHKYCDPNLGLRVQQKGTSETDTICTCEEGWHCTSEACESCVLHRSCS
PGFGVKQIATGVSDTICEPCPVGFFSNVSSAFEKCHPWTSCETKDLVVQQAGTNKTDVVCGP
QDRLRALVVIPIIFGILFAILLVLVFIKKVAKKPTNKAPHPKQEPQEINFPDDLPGSNTAAPVQE
TLHGCQPVTQEDGKESRISVQERQ
(CD40/CD40L + 20mer linker)
>SEQ ID.NO: 5
MVRLPLQCVLWGCLLTAVHPEHRRLDKIEDERNLHEDFVFMKTIQRCNTGERSLSLLNC
EEIKSQFEGFVKDIMLNKEETKKENSFEMQKGDQNPQIAAHVISEASSKTTSVLQWAEK
GYYTMSNNLVTLENGKQLTVKRQGLYYIYAQVTFCSNREASSQAPFIASLCLKSPGRFE
RILLRAANTHSSAKPCGQQSIHLGGVFELQPGASVFVNVTDPSQVSHGTGFTSFGLLKLG
GSGGGGSGGGGSGGGGSGGPPTACREKQYLINSQCCSLCQPGQKLVSDCTEFTETECLP
CGESEFLDTWNRETHCHQHKYCDPNLGLRVQQKGTSETDTICTCEEGWHCTSEACESCVLH
RSCSPGFGVKQIATGVSDTICEPCPVGFFSNVSSAFEKCHPWTSCETKDLVVQQAGTNKTDVV
CGPQDRLRALVVIPIIFGILFAILLVLVFIKKVAKKPTNKAPHPKQEPQEINFPDDLPGSNTAAP
VQETLHGCQPVTQEDGKESRISVQERQ
(mouse_CD40/CD40L + 12mer linker)
>SEQ ID.NO: 6
MVSLPRLCALWGCLLTAVHLHRRLDKVEEEVNLHEDFVFIKKLKRCNKGEGSLSLLNC
EEMRRQFEDLVKDITLNKEEKKENSFEMQRGDEDPQIAAHVVSEANSNAASVLQWAKK
GYYTMKSNLVMLENGKQLTVKREGLYYVYTQVTFCSNREPSSQRPFIVGLWLKPSSGS
ERILLKAANTHSSSQLCEQQSVHLGGVFELQAGASVFVNVTEASQVIHRVGFSSFGLLKL
GGGSGGGGSGGGGQCVTCSDKQYLHDGQCCDLCQPGSRLTSHCTALEKTQCHPCDSGE
FSAQWNREIRCHQHRHCEPNQGLRVKKEGTAESDTVCTCKEGQHCTSKDCEACAQHTPCIP
GFGVMEMATETTDTVCHPCPVGFFSNQSSLFEKCYPWTSCEDKNLEVLQKGTSQTNVICGLK
SRMRALLVIPVVMGILITIFGVFLYIKKVVKKPKDNEILPPAARRQDPQEMEDYPGHNTAAPVQ
ETLHGCQPVTQEDGKESRISVQERQVTDSIALRPLV
(mouse_CD40/CD40L + 14mer linker)
>SEQ ID.NO: 7
MVSLPRLCALWGCLLTAVHLHRRLDKVEEEVNLHEDFVFIKKLKRCNKGEGSLSLLNC
EEMRRQFEDLVKDITLNKEEKKENSFEMQRGDEDPQIAAHVVSEANSNAASVLQWAKK
GYYTMKSNLVMLENGKQLTVKREGLYYVYTQVTFCSNREPSSQRPFIVGLWLKPSSGS
ERILLKAANTHSSSQLCEQQSVHLGGVFELQAGASVFVNVTEASQVIHRVGFSSFGLLKL
GGGGSGGGGSGGGGGQCVTCSDKQYLHDGQCCDLCQPGSRLTSHCTALEKTQCHPCDS
GEFSAQWNREIRCHQHRHCEPNQGLRVKKEGTAESDTVCTCKEGQHCTSKDCEACAQHTP
CIPGFGVMEMATETTDTVCHPCPVGFFSNQSSLFEKCYPWTSCEDKNLEVLQKGTSQTNVIC
GLKSRMRALLVIPVVMGILITIFGVFLYIKKVVKKPKDNEILPPAARRQDPQEMEDYPGHNTAA
PVQETLHGCQPVTQEDGKESRISVQERQVTDSIALRPLV
(mouse_CD40/CD40L + 16mer linker)
>SEQ ID.NO: 8
MVSLPRLCALWGCLLTAVHLHRRLDKVEEEVNLHEDFVFIKKLKRCNKGEGSLSLLNC
EEMRRQFEDLVKDITLNKEEKKENSFEMQRGDEDPQIAAHVVSEANSNAASVLQWAKK
GYYTMKSNLVMLENGKQLTVKREGLYYVYTQVTFCSNREPSSQRPFIVGLWLKPSSGS
ERILLKAANTHSSSQLCEQQSVHLGGVFELQAGASVFVNVTEASQVIHRVGFSSFGLLKL
GGGSGGGGSGGGGSGGGQCVTCSDKQYLHDGQCCDLCQPGSRLTSHCTALEKTQCHPC
DSGEFSAQWNREIRCHQHRHCEPNQGLRVKKEGTAESDTVCTCKEGQHCTSKDCEACAQH
TPCIPGFGVMEMATETTDTVCHPCPVGFFSNQSSLFEKCYPWTSCEDKNLEVLQKGTSQTNV
ICGLKSRMRALLVIPVVMGILITIFGVFLYIKKVVKKPKDNEILPPAARRQDPQEMEDYPGHNT
AAPVQETLHGCQPVTQEDGKESRISVQERQVTDSIALRPLV
(mouse_CD40/CD40L + 18mer linker)
>SEQ ID.NO: 9
MVSLPRLCALWGCLLTAVHLHRRLDKVEEEVNLHEDFVFIKKLKRCNKGEGSLSLLNC
EEMRRQFEDLVKDITLNKEEKKENSFEMQRGDEDPQIAAHVVSEANSNAASVLQWAKK
GYYTMKSNLVMLENGKQLTVKREGLYYVYTQVTFCSNREPSSQRPFIVGLWLKPSSGS
ERILLKAANTHSSSQLCEQQSVHLGGVFELQAGASVFVNVTEASQVIHRVGFSSFGLLKL
GSGGGGSGGGGSGGGGSGGQCVTCSDKQYLHDGQCCDLCQPGSRLTSHCTALEKTQCH
PCDSGEFSAQWNREIRCHQHRHCEPNQGLRVKKEGTAESDTVCTCKEGQHCTSKDCEACA
QHTPCIPGFGVMEMATETTDTVCHPCPVGFFSNQSSLFEKCYPWTSCEDKNLEVLQKGTSQT
NVICGLKSRMRALLVIPVVMGILITIFGVFLYIKKVVKKPKDNEILPPAARRQDPQEMEDYPGH
NTAAPVQETLHGCQPVTQEDGKESRISVQERQVTDSIALRPLV
(mouse_CD40/CD40L + 20mer linker)
>SEQ ID.NO: 10
MVSLPRLCALWGCLLTAVHLHRRLDKVEEEVNLHEDFVFIKKLKRCNKGEGSLSLLNC
EEMRRQFEDLVKDITLNKEEKKENSFEMQRGDEDPQIAAHVVSEANSNAASVLQWAKK
GYYTMKSNLVMLENGKQLTVKREGLYYVYTQVTFCSNREPSSQRPFIVGLWLKPSSGS
ERILLKAANTHSSSQLCEQQSVHLGGVFELQAGASVFVNVTEASQVIHRVGFSSFGLLKL
GGSGGGGSGGGGSGGGGSGGGQCVTCSDKQYLHDGQCCDLCQPGSRLTSHCTALEKT
QCHPCDSGEFSAQWNREIRCHQHRHCEPNQGLRVKKEGTAESDTVCTCKEGQHCTSKDCE
ACAQHTPCIPGFGVMEMATETTDTVCHPCPVGFFSNQSSLFEKCYPWTSCEDKNLEVLQKG
TSQTNVICGLKSRMRALLVIPVVMGILITIFGVFLYIKKVVKKPKDNEILPPAARRQDPQEMED
YPGHNTAAPVQETLHGCQPVTQEDGKESRISVQERQVTDSIALRPLV

Further constructs for 4-1BBL/4-1BB and Ox40L/Ox40 may be based on the following Uniprot sequences in a manner substantially as described above for CD40L/CD40:

human 4-1BB: www.uniprot.org/uniprot/Q07011
human 4-1BBL: www.uniprot.org/uniprot/P41273
mouse 4-1BB: www.uniprot.org/uniprot/P20334
mouse 4-1BBL: www.uniprot.org/uniprot/P41274
human Ox40: www.uniprot.org/uniprot/P43489
human Ox40L: www.uniprot.org/uniprot/P23510
mouse Ox40: www.uniprot.org/uniprot/P47741
mouse Ox40L: www.uniprot.org/uniprot/P43488

What is claimed is:

1. A recombinant expression cassette comprising a promoter operably coupled to a segment that encodes the chimeric protein comprising, in sequence from N- to C-terminus, an extracellular portion of a TNF family member ligand coupled by a flexible linker to its corresponding TNF family member receptor.

2. The recombinant expression cassette of claim 1 further comprising a leader peptide that is coupled to the N-terminus of the extracellular portion of CD40L.

3. The recombinant expression cassette of claim 1 wherein the flexible linker has a length of between 4-25 amino acids, and optionally comprises a (GₙS)ₓ sequence.

4. The recombinant expression cassette of claim 1 wherein the TNF family member ligand is CD40L, 4-1BB ligand, or Ox40 ligand, and wherein the TNF family member receptor is CD40, 4-1BB, or Ox40.

5. The recombinant expression cassette of claim 1 wherein the TNF family member receptor is a human TNF family member receptor, and/or wherein the TNF family member receptor lacks a signal sequence.

6. The recombinant expression cassette of claim 1 having a sequence of any one of SEQ ID NO: 1-10.

7. The recombinant expression cassette of claim 1 further comprising a second segment that encodes a cytokine and/or at least a portion of at least one of a tumor associated antigen, a tumor specific antigen, and a tumor and patient specific neoepitope.

8. The recombinant expression cassette of claim 1 wherein the cassette is a RNA.

9. The recombinant expression cassette of claim 1 wherein the cassette is part of a viral expression vector.

10. A method of enhancing an immune reaction against an antigen, comprising:
transfecting an antigen presenting cell with a recombinant expression cassette comprising a promoter operably coupled to a segment that encodes the chimeric protein comprising, in sequence from N- to C-terminus, an extracellular portion of a TNF family member ligand coupled by a flexible linker to its corresponding TNF family member receptor;
contacting the transfected cell with the antigen or expressing the antigen in the transfected cell; and
upon contact or expression, contacting the transfected cell with at least one of a CD4+ T cell and a CD8+ T cell.

11. The method of claim 10 wherein the antigen presenting cell is a dendritic cell.

12. The method of claim 10 wherein the antigen presenting cell is transiently transfected.

13. The method of claim 10 wherein the antigen is a tumor and patient specific neoepitope, or at least a portion of a tumor associated antigen or a tumor specific antigen.

14. The method of claim 10 wherein the step of transfecting is performed ex vivo, and wherein the steps of contacting are performed in vivo.

15. The method of claim 10 wherein the immune reaction against the antigen is an immune reaction against a tumor or against a virus.

16. The method of claim 15 wherein the tumor is a solid tumor, or wherein the virus is an HIV virus.

17. A method of treating a tumor in a patient, comprising:
administering to the patient an antigen presenting cell transfected with a recombinant expression cassette comprising a promoter operably coupled to a segment that encodes the chimeric protein comprising, in sequence from N- to C-terminus, an extracellular portion of a TNF family member ligand coupled by a flexible linker to its corresponding TNF family member receptor, and wherein the transfected cell further expresses a tumor antigen.

18. The method of claim 17 wherein the transfecting is performed ex vivo.

19. The method of claim 17 wherein the tumor antigen is a tumor and patient specific neoepitope, or at least a portion of a tumor associated antigen or a tumor specific antigen.

20. The method of claim 17 wherein the antigen presenting cell is a dendritic cell, and wherein the recombinant expression cassette is an mRNA or part of an adenovirus.
